(19) United States
(12) Patent Application Publication
(10) Pub. No.: US 2006/0166955 A1
(43) Pub. Date: Jul. 27, 2006

(54) Production process for 16-dehydropregnenoneol and its analogs

(75) Inventors: Weisheng Tian, Shanghai (CN); Xin Xu, Shanghai (CN); Shanshan Liu, Shanghai (CN); Junwei Shen, Shanghai (CN); Xiujing Wu, Shanghai (CN)

Correspondence Address: VENABLE LLP, P.O. BOX 34385, WASHINGTON, DC, 20045-9998, US

(73) Assignee: SHANGHAI INSTITUTE OF ORGANIC CHEMISTRY, CHINESE ACADEMY OF SCIENCES, SHANGHAI (CN)

(21) Appl. No.: 10/561,164

(22) PCT Filed: Jun. 14, 2004
(86) PCT No.: PCT/CN04/00636

§ 371 Date: Dec. 16, 2005

(30) Foreign Application Priority Data
Jul. 16, 2003 (CN) 03141641.1

(51) Int. Cl. A61K 31/56; C07J 5/00

(52) U.S. Cl. 514/177; 552/600

(57) ABSTRACT

The present invention relates to a clean process for the degradation of steroidal sapogenin to produce 16-dehydropregnenolone and its analogs. The pure or the crude pseudo steroidal sapogenin, derived from steroidal sapogenin, dissolved in organic solvent, reacts with hydrogen peroxide with or without metal compound and acid as catalyst, and the crude products directly go through elimination and hydrolization in the presence of base to give 16-Dehydropregnenolone or its analog, accompanied with the other product 4R(or S)-methyl-5-hydroxy-pentate, which is converted to 4R(or S)-methyl-δ-pentyl lactone after acidification and extraction from the water layer. This technology improved the utilizing degree of steroidal sapogenin, improved the yield, and cleared up the chromium pollution in the former technique. In a word, the method disclosed in this invention is more suitable for manufacture.

### TECHNICAL FIELD

The invention relates to a clean process for the degradation of steroidal sapogenin to produce 16-dehydropregnenolone and its analogs.

### BACKGROUND ART

16-Dehydropregnenolone (3β-hydroxypregn-5(6),16(17)-diene-20-one)is the hydrolysate of 16-Dehydropregnenolone acetate which is called ‘diene’ in industry. Its analogs include 3β-hydroxy-5α-pregn-16(17)-ene-20-one, 3β-hydroxy-5β-pregn-16(17)-ene-20-one, 3β,12β-dihydroxy-5α-pregn-16(17)-ene-20-one, 3β,12α-dihydroxy-5α-pregn-16(17)-ene-20-one, 3β-hydroxy-5α-pregn-12,20-dione.

16-Dehydropregnenolone acetate and 3β-hydroxy-5α-pregn-16(17)-ene-20-one acetate are important intermediates of steroidal hormone drugs. The throughput of the two compounds is kiloton and several hundred ton in China each year, respectively.

But the technique of manufacturing the two compounds used today is still based on the old degradation method of steroidal sapogenin which was developed by Marker, an American chemist, in the 40′ last century (Marker: J. Am. Chem. Soc. 1940, 62, 3350. 1941, 63, 774. 1947, 69 2167). It can be described below: in acetic anhydride and acetic acid, under high pressure and at high temperature (above 200° C.), steroidal sapogenin is degradated to pseudo steroidal sapogenin, which is oxidized by chromate. After elimination, the corresponding 16-Dehydropregnenolone is obtained. The overall yield is about 60% for the three steps. Taking diosgenin for example, it can be outlined below:

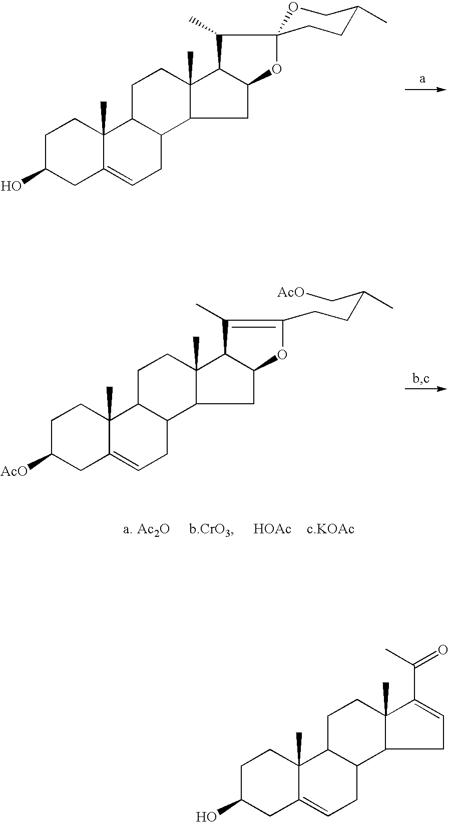

Though this method had been ameliorated successively, the shortcomings still exist. One of the shortcomings is the oxidation with chromate in the procedure, which caused serious environmental pollution. Therefor, Professor Weisheng Tian and his co-workers had opened out the research of how to utilize steroidal sapogenin rationally since 1991.

The present invention is the extending of Tian's prevenient inventions (Weisheng Tian, et al, Chinese patent, patent No: 96116304.6; Chinese patent, application No: 00127974.2; Chinese patent, application No: 01113196.9 etc).

### ABSTRACT OF THE INVENTION

In this invention, the pseudo steroidal sapogenin, degradated from steroidal sapogenin without purification, is oxidized with hydrogen peroxide (instead of chromate) in organic solvent with or without metal catalysts. After elimination and hydrolization, the corresponding 16-Dehydropregnenolone or its analog is obtained. Another product, 4R(or S)-methyl-pentyl lactone, is produced in the process. The mentioned steroidal sapogenin includes: diosgenin, tigogenin, sarsasapogenin, hecogenin, and other natural steroidal sapogenin. It also includes the analogs modified from natural steroidal. The structure of 16-dehydropregnenolone and its analogs can be outlined below:

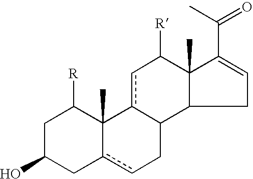

### DISCLOSURE OF THE INVENTION

The invention relates to a clean process for the degradation of steroidal sapogenin to produce 16-dehydropregnenolone and its analogs.

In this invention, the crude pseudo steroidal sapogenin, degradated from steroidal sapogenin, is oxidized with hydrogen peroxide in the presence of metal catalysts. After elimination and hydrolization, the corresponding 16-Dehydropregnenolone or its analog is obtained. Another product, 4R(or S)-methyl-pentyl lactone, is produced in the process.

In this invention, the pseudo steroidal sapogenin, degradated from steroidal sapogenin, is oxidized with hydrogen peroxide (instead of chromate) in organic solvent with or without metal catalysts. After elimination and hydrolization, the corresponding 16-Dehydropregnenolone or its analog is obtained. Another product, 4R(or S)-methyl-pentyl lactone, is produced in the process. For example:

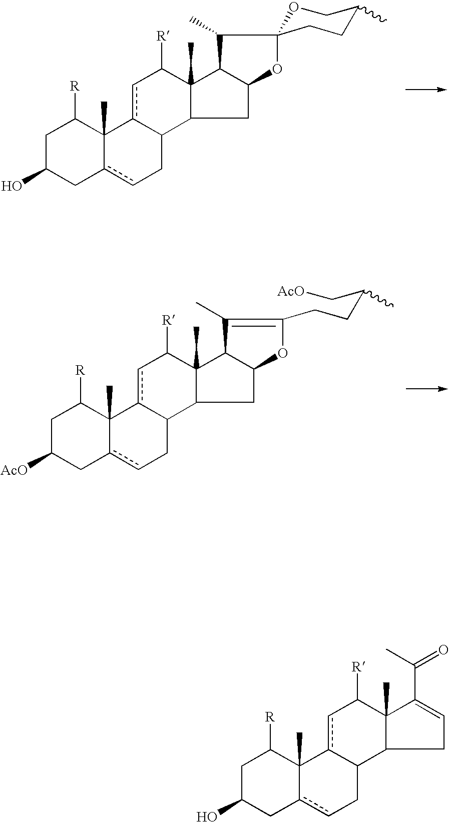

The operation of this invention is described below:

Firstly, steroidal sapogenin is degradated to pseudo steroidal sapogenin under high pressure according to known method. Then, the pseudo steroidal sapogenin went through oxidation, elimination and hydrolization. 16-Dehydropregnenolone or its analog is obtained, accompanied with 4R(or S)-methyl-δ-pentyl lactone.

This invention is different from the patent CN: 01113196.9. The product described in the patent CN: 01113196.9 is 16-Dehydropregnenolone acetate, while it is 16-Dehydropregnenolone in this invention.

The crude degradation product is dissolved in organic solvent, and then hydrogen peroxide, metal catalyst and acid are added. The molar ratio of pseudo steroidal sapogenin, hydrogen peroxide, metal catalyst and acid is 1:1.0-4.0:0.001-1:0-1, of which 1:1.5-2.5:0.005-0.02:0 is preferred. The reaction temperature is 0-80° C. The reaction time is 10 min to 24 hour. The reaction is monitored by chromatogram until the starting material disappeared. The mixture is refluxed with base for 0.5-2 hour to completely convert the unreacted 16-carbonoxyl-20-one to 16-Dehydropregnenolone or its analog, accompanied with the other product 4R(or S)-methyl-5-hydroxy-pentate. Remove part of the organic solvent under reduced pressure then add water to precipitate 16-Dehydropregnenolone or its analog. The water layer is acidified and extracted with organic solvent to give 4R(or S)-methyl-δ-pentyl lactone.

The mentioned steroidal sapogenin includes: diosgenin, tigogenin, sarsasapogenin, hecogenin, and other natural steroidal sapogenin. It also includes the analogs modified from natural steroidal sapogenin.

The mentioned steroidal sapogenin is of the structure:

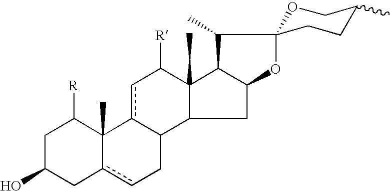

In which R or R′ is H or OH, C-5(6) and/or C-9(11) is C—C or C═C, C-25R or C-25S, and C-5 is 5α-H or 5β-H when C-5(6) is C—C.

The structure of the mentioned 16-dehydropregnenolone and its analogs can be outlined below:

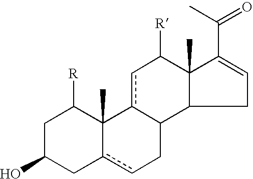

In which R or R′ is H or OH, C-5(6) and/or C-9(11) is C—C or C═C, and C-5 is 5α-H or 5β-H when C-5(6) is C—C.

The mentioned metal catalyst include: tungstic oxide (WO₃), tungstate, vanadic acid, vanadate, vanadyl acetylacetonate, molybdic anhydride (MoO₃), molybdate, phosphomolybdate, heteropolyacid, heteropolyate.

The mentioned acid include carboxylic acid, sulfonic acid and inorganic acid, where the carboxylic acid is preferable to be acetic acid, formic acid, propionic acid, butyric acid, benzoic acid, phthalic acid and isophthalic acid, the sulfonic acid is preferable to be benzenesulfonic acid and p-toluene sulphonic acid, and the inorganic acid is preferable to be sulfuric acid (H₂SO₄), phosphoric acid (H₃PO₄) and phosphorous acid (H₃PO₃).

The mentioned organic solvent include dihalogen methane, trihalogen methane, dichloroethane, ethanol (EtOH), butanol (BuOH), t-butanol (t-BuOH), dimethyl sulphoxide (DMSO), N,N-dimethylformamide (DMF), acetone, butanone, cyclohexanone, acetonitrile, ethyl acetate and acetic acid.

The mentioned base include: hydroxid, carbonate and bicarbonate, preferably to be sodium hydroxide (NaOH), potassium hydroxide (KOH), lithium hydroxide (LiOH), cesium hydroxide (CsOH), sodium carbonate (Na₂CO₃), potassium carbonate (K₂CO₃), lithium carbonate (Li₂CO₃), cesium carbonate (Cs₂CO₃), sodium bicarbonate (NaHCO₃) and potassium bicarbonate (KHCO₃).

This invention had been verified in hundred-gram scale for several times. This technology improved the utilizing degree of steroidal sapogenin, improved the yield, and cleared up the chromium pollution in former technique. In a word, the method disclosed in this invention is more suitable for manufacture.

### EXAMPLES

The invention is illustrated below with reference to the following examples. Other features of the invention will become apparent in the course of the following descriptions of exemplary embodiments that are given for illustration of the invention and are not intended to be limiting thereof.

### Example 1

### Degradation of sarsasapogenin to 3β-hydroxy-5β-pregn-16(17)-ene-20-one and 4S-methyl-δ-pentyl lactone

10 g of sarsasapogenin, dissolved in acetic acid and acetic anhydride, was kept in the pressure kettle at 200° C. for one hour, then 3.3 mg of Na₂WO₄.2H₂O (0.01 mmol) and 5 ml of hydrogen peroxide (30% H₂O) were added, and this mixture was stirred for 2 hours at 80° C. The low boilers were removed under reduced pressure and the residue was dissolved in 50 ml of EtOH, and refluxed for 2 hours with 5% LiOH. The mixture was concentrated, diluted with water, and filtrated to get 6.3 g of 3β-hydroxy-5β-pregn-16(17)-ene-20-one in 84% yield. m.p. 186-8° C., ¹H-NMR (300 MHz, CDCl₃) δ(ppm): 6.61 (dd, J=1.3 Hz, 1H, 16-H), 3.5 (m, 1H, 3-H), 2.26 (s, 3H, CH₃CO—, 21-H), 0.84 (s,3H, 18-H), 0.88 (s, 3H, 19-H). MS (m/z, %): 316 (M⁺), 301 (M⁺-CH₃), 283 (M⁺-CH₃— H₂O), 159, 145, 115, 105, 91, 43. The water layer was acidified and extracted with organic solvent to give 2.1 g of 4S-methyl-δ-pentyl lactone in 80% yield. [α]²⁰_{D}=−13° (c 0.8, CHCl₃), IR(v): 2950, 1730, 1340, 1210, 1190, 1040 cm⁻¹. ¹H-NMR (300 MHz, CDCl₃) δ(ppm): 0.96 (d, 3H, J=6.6 Hz), 1.88-2.06 (m, 2H), 1.43-1.56 (m, 1H), 3.83-3.90 (m, 1H), 4.23-4.29 (m, 1H). MS (m/z, %): 115 (M⁺+1), 114 (M⁺), 109, 56, 42.

### Example 2

### Degradation of sarsasapogenin to 3β-hydroxy-5β-pregn-16(17)-ene-20-one and 4S-methyl-δ-pentyl lactone:

100 g of sarsasapogenin, dissolved in acetic acid and acetic anhydride, was kept in the pressure kettle at 200° C. for one hour, then the low boilers were removed under reduced pressure and the residue was dissolved in 500 ml of BuOH. 23 mg of WO₃ (0.01 mmol), 10 g of isophthalic acid and 50 ml of hydrogen peroxide (30% H₂O) were added, and this mixture was stirred for 2 hours at 80° C. After 5% KOH was added, it was kept on refluxing for 2 hours. The mixture was concentrated, diluted with water, and filtrated to get 66 g of 3β-hydroxy-5β-pregn-16(17)-ene-20-one in 88% yield. The water layer was acidified and extracted with organic solvent to give 22 g of 4S-methyl-δ-pentyl lactone in 84% yield.

### Example 3

### Degradation of sarsasapogenin to 3β-hydroxy-5β-pregn-16(17)-ene-20-one and 4S-methyl-δ-pentyl lactone:

100 g of sarsasapogenin, dissolved in acetic acid and acetic anhydride, was kept in the pressure kettle at 200° C. for one hour, then the low boilers were removed under reduced pressure and the residue was dissolved in 500 ml of BuOH. 23 mg of WO₃ (0.01 mmol), 1 g of isophthalic acid and 50 ml of hydrogen peroxide (30% H₂O) were added, and this mixture was stirred for 2 hours at 80° C. After 5% KOH was added, it was kept on refluxing for 2 hours. The mixture was concentrated, diluted with water, and filtrated to get 60 g of 3β-hydroxy-5β-pregn-16(17)-ene-20-one in 80% yield. The water layer was acidified and extracted with organic solvent to give 22 g of 4S-methyl-δ-pentyl lactone in 84% yield.

### Example 4

### Degradation of diosgenin to 16-dehydropregnenolone and 4R-methyl-δ-pentyl lactone

100 g of diosgenin, dissolved in acetic acid and acetic anhydride, was kept in the pressure kettle at 200° C. for one hour, then 3.3 mg of (NH₄)₂MoO₄ (0.01 mmol), 5 g of benzoic acid and 50 ml of hydrogen peroxide (30% H₂O) were added, and this mixture was stirred for 2 hours at 80° C. The low boilers were removed under reduced pressure and the residue was dissolved in 500 ml of cyclohexatone, and refluxed for 2 hours with 5% CsOH. The mixture was concentrated, diluted with water, and filtrated to get 64 g of 16-dehydropregnenolone in 84% yield. m.p. 168-170° C., ¹H-NMR (300 MHz, CDCl₃)δ(ppm): 6.72 (dd, J=1.3 Hz, 1H, 16-H), 5.38 (d, J=4Hz, 1H, 6-H), 2.26 (s, 3H, CH₃CO—, 21-H), 0.85 (s, 3H, 18-H), 0.88 (s, 3H, 19-H). MS (m/z, %): 314 (M⁺), 299 (M⁺-CH₃), 281 (M⁺-CH₃—H₂O), 253, 239, 229, 203, 159, 145, 115, 105, 91, 43. The water layer was acidified and extracted with organic solvent to give 22 g of 4R-methyl-δ-pentyl lactone in 80% yield. b.p. 83-89° C./15 mmHg, [α]²⁰_{D}=+13.6° (c 0.9 CHCl₃), IR(v): 2950, 1730, 1340, 1210, 1190, 1040 cm⁻¹. ¹H-NMR (300 MHz, CDCl₃) δ(ppm): 0.96 (d, 3H, J=6.6 Hz), 1.88-2.06 (m, 2H), 1.43-1.56 (m, 1H), 3.83-3.90 (m, 1H), 4.23-4.29 (m, 1H). MS(m/z, %): 115 (M⁺+1), 114 (M⁺), 109, 56, 42.

### Example 5

### Degradation of diosgenin to 16-dehydropregnenolone and 4R-methyl-δ-pentyl lactone

100 g of diosgenin, dissolved in acetic acid and acetic anhydride, was kept in the pressure kettle at 200° C. for one hour, then the low boilers were removed under reduced pressure and the residue was dissolved in 500 ml of EtOH. 182 mg of ammonium phosphomolybdate ((NH₄)₃[P(Mo₁₂O₄₀)].6H₂O, 0.1 mmol) and 50 ml of hydrogen peroxide (30% H₂O) were added, and this mixture was stirred for 2 hours at 80° C. After NaHCO₃ was added, it was kept on refluxing for 2 hours. The mixture was concentrated, diluted with water, and filtrated to get 72 g of 16-dehydropregnenolone in 95% yield. The water layer was acidified and extracted with organic solvent to give 24 g of 4R-methyl-δ-pentyl lactone in 88% yield.

### Example 6

### Degradation of diosgenin to 16-dehydropregnenolone and 4R-methyl-δ-pentyl lactone

100 g of diosgenin, dissolved in acetic acid and acetic anhydride, was kept in the pressure kettle at 200° C. for one hour, then the low boilers were removed under reduced pressure and the residue was dissolved in 500 ml of EtOH. 3.48 g of VO(acac)₂ and 50 ml of hydrogen peroxide (30% H₂O) were added, and this mixture was stirred for 2 hours at 80° C. After NaHCO₃ was added, it was kept on refluxing for 2 hours. The mixture was concentrated, diluted with water, and filtrated to get 71 g of 16-dehydropregnenolone in 93% yield. The water layer was acidified and extracted with organic solvent to give 24 g of 4R-methyl-δ-pentyl lactone in 88% yield.

### Example 7

### Degradation of diosgenin to 16-dehydropregnenolone and 4R-methyl-δ-pentyl lactone

10 g of diosgenin, dissolved in acetic acid and acetic anhydride, was kept in the pressure kettle at 200° C. for one hour, then the low boilers were removed under reduced pressure and the residue was dissolved in 50 ml of dichloromethane. 18 mg of (NH₄)₃[P(Mo₁₂O₄₀)].6H₂O (0.1 mmol) and 5 ml of hydrogen peroxide (30% H₂O) were added, and this mixture was refluxed for 2 hours. After K₂CO₃ was added, it was kept on refluxing for 2 hours. The mixture was concentrated, diluted with water, and filtrated to get 7.2 g of 16-dehydropregnenolone in 95% yield. The water layer was acidified and extracted with organic solvent to give 2.4 g of 4R-methyl-δ-pentyl lactone in 88% yield.

### Example 8

### Degradation of diosgenin to 16-dehydropregnenolone and 4R-methyl-δ-pentyl lactone

100 g of diosgenin, dissolved in acetic acid and acetic anhydride, was kept in the pressure kettle at 200° C. for one hour, then the low boilers were removed under reduced pressure and the residue was dissolved in 500 ml of t-BuOH. 23 mg of WO₃, 2 ml of H₃PO₄ and 50 ml of hydrogen peroxide (30% H₂O) were added, and this mixture was stirred for 2 hours at 80° C. After KOH was added, it was kept on refluxing for 2 hours. The mixture was concentrated, diluted with water, and filtrated to get 70 g of 16-dehydropregnenolone in 92% yield. The water layer was acidified and extracted with organic solvent to give 23 g of 4R-methyl-δ-pentyl lactone in 84% yield.

### Example 9

### Degradation of tigogenin to 3β-hydroxy-5α-pregn-16(17)-ene-20-one and 4R-methyl-δ-pentyl lactone

100 g of tigogenin, dissolved in acetic acid and acetic anhydride, was kept in the pressure kettle at 200° C. for one hour, then the low boilers were removed under reduced pressure and the residue was dissolved in 500 ml of BuOH. 200 mg of Na₃[P(W₁₂O₄₀)] and 50 ml of hydrogen peroxide (30% H₂O) were added, and this mixture was stirred for 2 hours at 80° C. After NaOH was added, it was kept on refluxing for 2 hours. The mixture was concentrated, diluted with water, and filtrated to get 70 g of 3β-hydroxy-5α-pregn-16(17)-ene-20-one in 92% yield. m.p. 207-9° C., [α]²⁰_{D}=+51° (c 0.9 CHCl₃), ¹H-NMR (300 MHz, CDCl₃)δ(ppm): 6.59 (dd, J=1.3 Hz, 1H, 16-H), 3.45 (m, 1H, 3-H), 2.26 (s, 3H, CH₃CO—, 21-H), 0.83 (s, 3H, 18-H), 0.89 (s, 3H, 19-H). MS (m/z, %): 316 (M⁺), 301 (M⁺-CH₃), 283 (M⁺-CH₃—H₂O), 159, 145, 115, 105, 91, 43. The water layer was acidified and extracted with organic solvent to give 21 g of 4R-methyl-δ-pentyl lactone in 80% yield.

### Example 10

### Degradation of tigogenin to 3β-hydroxy-5α-pregn-16(17)-ene-20-one and 4R-methyl-δ-pentyl lactone

10 g of tigogenin, dissolved in acetic acid and acetic anhydride, was kept in the pressure kettle at 200° C. for one hour, then the low boilers were removed under reduced pressure and the residue was dissolved in 50 ml of DMF. 48 mg of Na₂MoO₄.2H₂O (0.2 mmol), 0.1 ml of H₂SO₄ and 5 ml of hydrogen peroxide (30% H₂O) were added, and this mixture was stirred for 2 hours at 80° C. After KOH was added, it was kept on stirring for 2 hours at 80° C. The mixture was diluted with water and filtrated to get 6.8 g of 3β-hydroxy-5α-pregn-16(17)-ene-20-one in 90% yield. The water layer was acidified and extracted with organic solvent to give 2.3 g of 4R-methyl-δ-pentyl lactone in 84% yield.

### Example 11

### Degradation of tigogenin to 3β-hydroxy-5α-pregn-16(17)-ene-20-one and 4R-methyl-δ-pentyl lactone

100 g of tigogenin, dissolved in acetic acid and acetic anhydride, was kept in the pressure kettle at 200° C. for one hour, then 186 mg of H₇[(PMo₂O₇)₆].xH₂O (0.1 mmol) and 50 ml of hydrogen peroxide (30% H₂O) were added, and this mixture was stirred for 2 hours at 80° C. The low boilers were removed under reduced pressure and the residue was dissolved in 500 ml of EtOH containing 5% KOH, and refluxed for 2 hours. The mixture was concentrated, diluted with water, and filtrated to get 65 g of 3β-hydroxy-5α-pregn-16(17)-ene-20-one in 86% yield. The water layer was acidified and extracted with organic solvent to give 22 g of 4R-methyl-δ-pentyl lactone in 80% yield.

### Example 12

### Degradation of rockogenin to 3β,12β-dihydroxy-5α-pregn-16(17)-ene-20-one and 4R-methyl-δ-pentyl lactone

100 g of rockogenin, dissolved in acetic acid and acetic anhydride, was kept in the pressure kettle at 200° C. for one hour, then the low boilers were removed under reduced pressure and the residue was dissolved in 500 ml of BuOH. 30 mg of V₂O₅(0.2 mmol), 1 ml of phosphorous acid and 50 ml of hydrogen peroxide (30% H₂O) were added, and this mixture was stirred for 2 hours at 80° C. After KOH was added, it was kept on refluxing for 2 hours. The mixture was concentrated, diluted with water, and filtrated to get 70 g of 3β,12β-dihydroxy-5α-pregn-16(17)-ene-20-one in 91% yield. m.p. 203-205° C., [α]_{D}²⁵=+2.0 (c=1.00 CHCl₃),IR(v): 1645, 1580 cm⁻¹, ¹H-NMR (300 MHz, CDCl₃) δ(ppm): 6.90 (m, 1H, 16-H), 2.37 (s, 3H, CH₃CO—, 21-H), 0.87 (s, 3H, 18-H), 0.82 (s, 3H, 19-H). The water layer was acidified and extracted with organic solvent to give 23 g of 4R-methyl-δ-pentyl lactone in 84% yield.

### Example 13

### Degradation of rockogenin to 3β,12β-dihydroxy-5α-pregn-16(17)-ene-20-one and 4R-methyl-δ-pentyl lactone

100 g of rockogenin, dissolved in acetic acid and acetic anhydride, was kept in the pressure kettle at 200° C. for one hour, then the low boilers were removed under reduced pressure and the residue was dissolved in 500 ml of BuOH. 30 mg of MoO₃, 1 ml of phosphorous acid and 50 ml of hydrogen peroxide (30% H₂O) were added, and this mixture was stirred for 3 hours at 80° C. After KOH was added, it was kept on refluxing for 2 hours. The mixture was concentrated, diluted with water, and filtrated to get 70 g of 3β,12β-dihydroxy-5α-pregn-16(17)-ene-20-one in 91% yield. The water layer was acidified and extracted with organic solvent to give 23 g of 4R-methyl-δ-pentyl lactone in 84% yield.

### Example 14

### Degradation of rockogenin to 3β,12β-dihydroxy-5α-pregn-16(17)-ene-20-one and 4R-methyl-δ-pentyl lactone

100 g of rockogenin, dissolved in acetic acid and acetic anhydride, was kept in the pressure kettle at 200° C. for one hour, then the low boilers were removed under reduced pressure and the residue was dissolved in 500 ml of BuOH. 30 mg of WO₃(0.2 mmol), 1 ml of phosphorous acid and 50 ml of hydrogen peroxide (30% H₂O) were added, and this mixture was stirred for 2 hours at 80° C. After KOH was added, it was kept on refluxing for 2 hours. The mixture was concentrated, diluted with water, and filtrated to get 71 g of 3β,12β-dihydroxy-5α-pregn-16(17)-ene-20-one in 92% yield. The water layer was acidified and extracted with organic solvent to give 23 g of 4R-methyl-δ-pentyl lactone in 84% yield.

### Example 15

### Degradation of rockogenin to 3β,12β-dihydroxy-5α-pregn-16(17)-ene-20-one and 4R-methyl-δ-pentyl lactone

10 g of rockogenin, dissolved in acetic acid and acetic anhydride, was kept in the pressure kettle at 200° C. for one hour, then the low boilers were removed under reduced pressure and the residue was dissolved in 50 ml of DMSO. 24 mg of Na₂MoO₄.2H₂O (0.1 mmol), 1 ml of H₃PO₄ and 5 ml of hydrogen peroxide (30% H₂O) were added, and this mixture was stirred for 2 hours at 80° C. After Li₂CO₃ was added, it was kept on stirring for 2 hours at 80° C. The mixture was diluted with water and filtrated to get 7.2 g of 3β,12β-dihydroxy-5α-pregn-16(17)-ene-20-one in 94% yield. The water layer was acidified and extracted with organic solvent to give 2.4 g of 4R-methyl-δ-pentyl lactone in 88% yield.

### Example 16

### Degradation of rockogenin to 3β,12β-dihydroxy-5α-pregn-16(17)-ene-20-one and 4R-methyl-δ-pentyl lactone

10 g of rockogenin, dissolved in acetic acid and acetic anhydride, was kept in the pressure kettle at 200° C. for one hour, then the low boilers were removed under reduced pressure and the residue was dissolved in 50 ml of DMSO. 24 mg of Na₂MoO₄.2H₂O (0.1 mmol), 1 g of butyric acid and 5 ml of hydrogen peroxide (30% H₂O) were added, and this mixture was stirred for 2 hours at 80° C. After Li₂CO₃ was added, it was kept on stirring for 2 hours at 80° C. The mixture was diluted with water and filtrated to get 7.2 g of 3β,12β-dihydroxy-5α-pregn-16(17)-ene-20-one in 94% yield. The water layer was acidified and extracted with organic solvent to give 2.4 g of 4R-methyl-δ-pentyl lactone in 88% yield.

What is claimed is:

1. A clean technology of producing 16-dehydropregnenolone and its analogs, that is, the pure or the crude pseudo steroidal sapogenin, derived from steroidal sapogenin, dissolved in organic solvent, reacts with hydrogen peroxide for 0.5-24 h at 0-80° C. with/without metal compound and acid as catalyst, wherein the molar ratio of pseudo steroidal sapogenin, hydrogen peroxide, metal catalyst and acid is 1:1.0-4.0:0.001-1:0-1, of which 1:1.5-2.5:0.005-0.02:0 is preferred, and the base is added to the mixture and then the mixture is kept at 0-100° C. or in reflux for 0.5-2 hour to give 16-Dehydropregnenolone or its analog, accompanied with the other product 4R(or S)-methyl-5-hydroxy-pentate, which is converted to 4R(or S)-methyl-δ-pentyl lactone after acidification and extraction from the water layer.
The mentioned steroidal sapogenin is of the structure: 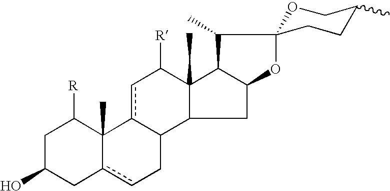
in which R or R′ is H or OH, C-5(6) and/or C-9(11) is C—C or C═C, C-25R or C-25S, and C-5 is 5α-H or 5β-H when C-5(6) is C—C.
The structure of the mentioned 16-dehydropregnenolone and its analogs can be outlined below: 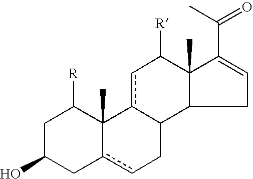
in which R or R′ is H or OH, C-5(6) and/or C-9(11) is C—C or C═C, and C-5 is 5α-H or 5β-H when C-5(6) is C—C.
The mentioned metal catalyst include: tungstic oxide, tungstate, vanadic acid, vanadate, vanadyl acetylacetonate, molybdic anhydride, molybdate, phosphomolybdate, heteropolyacid and heteropolyate.
The mentioned acid include carboxylic acid, sulfonic acid and inorganic acid, where the carboxylic acid is preferable to be acetic acid, formic acid, propionic acid, butyric acid, benzoic acid, phthalic acid and isophthalic acid, the sulfonic acid is preferable to be benzenesulfonic acid and p-toluene sulphonic acid, and the inorganic acid is preferable to be sulfuric acid, phosphoric acid and phosphorous acid.
The mentioned organic solvent include dihalogen methane, trihalogen methane, dichloroethane, ethanol, butanol, t-butanol, dimethyl sulphoxide, N,N-dimethylformamide, acetone, butanone, cyclohexanone, acetonitrile, ethyl acetate and acetic acid.
The mentioned base include: hydroxid, carbonate and bicarbonate, preferably to be sodium hydroxide, potassium hydroxide, lithium hydroxide, cesium hydroxide, sodium carbonate, potassium carbonate, lithium carbonate, cesium carbonate, sodium bicarbonate and potassium bicarbonate.

2. A process as defined in claim 1, wherein the steroidal sapogenin is diosgenin, tigogenin, sarsasapogenin, hecogenin, the other natural steroidal sapogenin or the analogs modified from natural steroidal sapogenin.

3. A process as defined in claim 1, wherein the molar ratio of pseudo steroidal sapogenin, hydrogen peroxide, metal catalyst and acid is 1:1.0-4.0:0.001-1:0-1, of which 1:1.5-2.5:0.005-0.02:0 is preferred.

4. A process as defined in claim 1, wherein 16-Dehydropregnenolone or its analog is obtained as precipitate after water was added to the reaction mixture and the water layer is acidified and extracted with organic solvent to give 4R(or S)-methyl-δ-pentyl lactone.
